(19) United States
(12) Patent Application Publication
(10) Pub. No.: US 2016/0354158 A1
(43) Pub. Date: Dec. 8, 2016

(54) METHODS AND SYSTEMS FOR VIRTUAL VENOUS IMAGING

(71) Applicant: PACESETTER, INC., Sylmar, CA (US)

(72) Inventors: Hoda Razavi, San Jose, CA (US); Kyungmoo Ryu, Palmdale, CA (US)

(21) Appl. No.: 14/732,371

(22) Filed: Jun. 5, 2015

(51) Int. Cl. A61B 34/20; A61B 5/0402; A61B 5/06; A61B 90/00

(52) U.S. Cl. CPC A61B 34/20; A61B 90/37; A61B 5/0402; A61B 5/066; A61B 2090/3762; A61B 2034/2048

(57) ABSTRACT

Methods and systems are provided for generating a virtual venous image. The methods and systems utilize an intravascular mapping tool configured to be inserted into a venous structure of an object of interest. The methods and systems further acquire a plurality of position data points at predetermined intervals corresponding to a position of the intravascular mapping tool within the venous structure, and adjust each position data point based on at least one of a cardiac motion or a respiratory motion of the object of interest. The methods and systems also display a travelled path of the intravascular mapping tool on the display based on the adjusted position data points.

### FIELD OF THE INVENTION

Embodiments of the present disclosure generally relate to methods and systems for virtual venous image, and more particularly for generating a virtual venography and/or angiography.

### BACKGROUND OF THE INVENTION

Cardiovascular navigation systems (CNS) provide real-time position and orientation information in relation to a part of the cardiovascular system, such as, the heart based on sensors placed at various locations within the cardiovascular system. The CNS may be integrated with a fluoroscopic (or other diagnostic) imaging system and track the sensors continuously within an imaging volume defined by the fluoroscopic system, on both live and recorded background diagnostic images.

CNS rely on one or more venograms or angiography acquired using, for example, the fluoroscopic imaging system to map the veins of the cardiovascular system for the imaging volume. Both the venogram and angiography are invasive procedures. For example, during both procedures, a catheter, such as a balloon catheter, continuously injects a contrast dye to fill the venous anatomy in a retrograde fashion. The contrast dye may be harmful in patients, notably for patients with kidney issues, for example, suffering from renal disease. Further, both procedures may be limited being dependent on the distribution of the contrast dye. For example, the contrast dye may note reach all branches of the cardiovascular system of the imaging volume or the contrast dye may be too diluted in certain branches, leading to partial visualization of the venous structure. Additionally, venograms expose the patient to ionizing radiation. For example, venograms are acquired with a higher-dose fluoroscopy protocol with a higher frame rate to enable visualization of smaller branches of the cardiovascular system. Thus, a need remains for improved methods and system for venous imaging.

### SUMMARY

In accordance with an embodiment herein, a method is provided for generating a virtual venous image for cardiovascular navigation system. The method obtains a medical image of a venous structure within an object of interest. The method further acquires a plurality of position data points at predetermined intervals corresponding to a position of an intravascular mapping tool within the venous structure, and group the position data points into phase intervals corresponding to at least one of a cardiac cycle or a respiratory cycle exhibited by object of interest. The method further generates a dynamic travel path of the intravascular mapping tool for a select phase interval based from the position data points, and displays the dynamic travel path overlaid on the medical image.

In an embodiment, a system for generating a virtual venous image. The system comprises a display and an intravascular mapping tool configured to be inserted into a venous structure of an object of interest. The system also includes a data storage configured to store a plurality of position data points collected by the intravascular mapping tool. Each position data point corresponds to a position of the intravascular mapping tool at a predetermined interval. The system also includes a processor configured to obtain a medical image of the venous structure, group the position data points into phase intervals corresponding to at least one of a cardiac cycle or a respiratory cycle of the object of interest, and generates a dynamic travel path of the intravascular mapping tool for a select phase interval based from the position data points. The processor is also configured to display the dynamic travel path overlaid on the medical image on the display.

### BRIEF DESCRIPTION OF THE DRAWINGS

FIG. 1 illustrates a cardiovascular navigation system for use in imaging an anatomical region and to acquire position data points, in accordance an embodiment herein.

FIG. 2 illustrates a method performed in accordance with embodiments herein for generating a virtual venous image.

FIG. 3 illustrates an intravascular mapping tool traversing within a venous structure, in accordance with an embodiment herein.

FIG. 4 illustrates timing diagrams showing cardiac physiologic measurements and position data points utilizing an intravascular mapping tool, in accordance with an embodiment herein.

FIG. 5 illustrates a timing diagram showing position data points grouped into phase intervals, in accordance with an embodiment herein.

FIG. 6A illustrates a two dimensional flourogram corresponding to an anterio-posterior angle overlaid with landmark icons, in accordance with an embodiment herein.

FIG. 6B illustrates a two dimensional flourogram corresponding to a left anterior oblique angle overlaid with landmark icons, in accordance with an embodiment herein.

FIG. 7A illustrates the two dimensional flourograms of FIG. 6A overlaid with a dynamic point-cloud of position data points, in accordance with an embodiment herein.

FIG. 7B illustrates the two dimensional flourograms of FIG. 6B overlaid with a dynamic point-cloud of position data points, in accordance with an embodiment herein.

FIG. 8A illustrates the two dimensional flourogram of FIG. 6A overlaid with directional vectors, in accordance with an embodiment herein.

FIG. 8B illustrates the two dimensional flourogram of FIG. 6B overlaid with directional vectors, in accordance with an embodiment herein.

FIG. 9A illustrates a two dimensional flourogram overlaid with a dynamic point-cloud of position data points, in accordance with an embodiment herein.

FIG. 9B illustrates a two dimensional flourogram overlaid with the dynamic point-cloud of position data points of FIG. 9A, in accordance with an embodiment herein.

FIG. 10A illustrates the two dimensional flourogram of FIG. 6A overlaid with tubular boundaries, in accordance with an embodiment herein.

FIG. 10B illustrates the two dimensional flourogram of FIG. 6B overlaid with tubular boundaries, in accordance with an embodiment herein.

FIG. 11 illustrates a system for analyzing motion data in accordance with an embodiment.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

Various embodiments described herein include a method and/or system to generate a virtual venous image, such as a virtual venogram without the use of contrast dye and/or live fluoroscopy. Additionally or alternatively, various embodiments described herein may be used for contrast-less and fluoroless angiography, such as a virtual angiography. In at least one embodiment, the virtual venous image may be generated by flagging or identifying positions of an intravascular mapping tool (e.g., guidewire, catheter) within the venous anatomy.

For example, a user (e.g., physician, clinician) may probe and/or traverse the intravascular mapping tool through and/or around the venous anatomy, while concurrently acquiring position measurements of the intravascular mapping tool. The position measurements or location of the intravascular mapping tool may be acquired automatically at pre-defined intervals (e.g., sampling frequency). Additionally or alternatively, the position measurements may be compensated and/or adjusted for patient movement, respiratory motion, cardiac motion, or the like. The position measurements may be recorded, for example on a data storage device.

The position measurements may be overlaid on a medical image and/or pre-recorded loops of medical images, such as a looped medical image, on a display of a three-dimensional (3D) space, a two-dimensional (2D) projection, or the like. Based on a compilation of the position measurements acquired over time, a travelled path of the intravascular mapping tool is formed that further corresponds to a structure and/or path of the venous anatomy. For example, the position measurements may form a dynamic point-cloud visualization of the travelled path of the intravascular mapping tool representing the interior of the vascular anatomy. Optionally, the dynamic point-cloud may be used to create tubular boundaries corresponding to the structure of the venous anatomy such that the venous structure is visualized on the display. Optionally, one or more anatomical labels may be automatically generated indicating portions of the venous structure (e.g., the main coronary sinus, side branches). A technical effect of the anatomical labels provides flexibility to the user in determining a representation of the venous anatomy represented by the position measurements.

Optionally, a portion and/or subset of the position measurements may be overlaid on the medical image. The position measurements may be selected based on a predetermined interval (e.g., time, distance, velocity) and/or acquired manually by the user. For example, the user may operate a user interface (e.g., a touchscreen, clicking a button) to select the position measurements of the intravascular mapping tool while traversing the venous structure to be overlaid on the medical image. The selected position measurements may be shown in the form of landmark icons on the display.

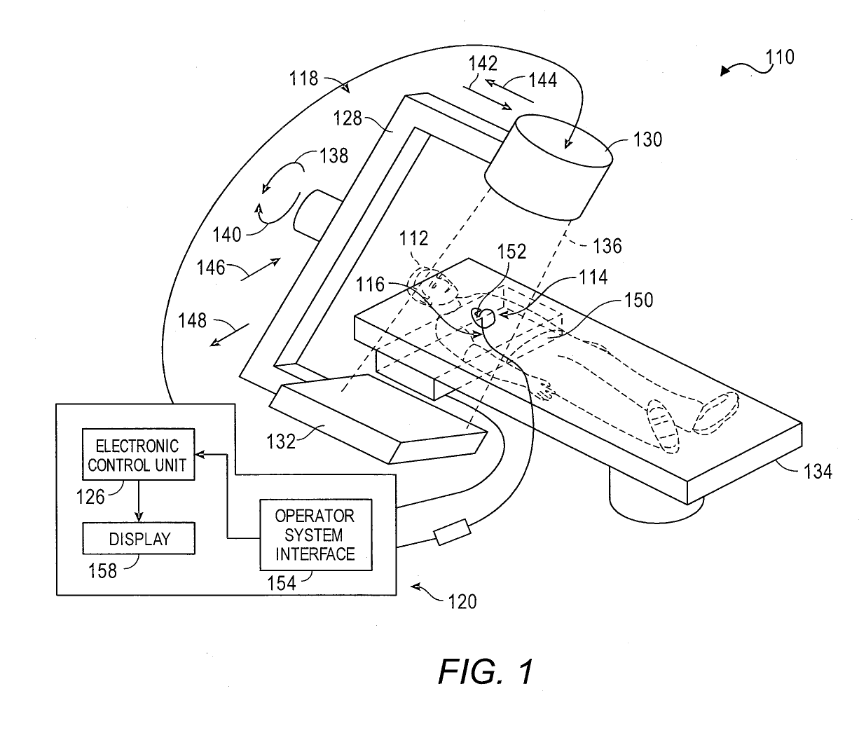

FIG. 1 illustrates a cardiovascular navigation system (CNS) 110, of an embodiment, for use in imaging an anatomical region of a patient 112, such as, a heart 114, upper torso of the patient, lower torso of the patient, or the like. A medical tool 116 is placed within the anatomical region, for example, the medical tool 116 may be configured to be inserted into a venous structure (e.g., coronary sinus) of the patient 112. The medical tool 116 may be for example, a guidewire, a mapping catheter or a catheter generally described or shown in U.S. Pat. No. 7,881,769, which is expressly incorporated herein by reference. The medical tool 116 may include one or more position sensors 152. The sensors 152 may be attached to the distal or proximal end of the medical tool 116, or any point in between. Optionally, the medical tool 116 may include additional sensors, such as motion sensors, electrical sensors, electrophysiological sensors, position sensors, hall sensors, or the like. The sensors 152 may transmit measurements (e.g., magnetic field measurements, electrical potential, motion, position) to an electronic control unit (ECU) 126 of a navigation system 120.

The navigation system 120 is provided to determine the position and/or orientation of the medical tool 116 within the body of the patient 112. In the illustrated embodiment, the navigation system 120 comprises a magnetic navigation system in which magnetic fields are generated in the anatomical region and the position sensors 152 associated with the medical tool 116 generate an output that is responsive to the position of the sensors within the magnetic field. The navigation system 120 may comprise, for example, the systems generally shown and described in, for example, U.S. Pat. Nos. 6,233,476; 7,197,354; 7,386,339; and 7,505,809 all of which are expressly incorporated by reference in their entirety. Although a magnetic navigation system is shown in the illustrated embodiment, it should be understood that the embodiments could find use with a variety of navigation systems including those based on the creation and detection of axes specific electric fields. The navigation system 120 may include a transmitter assembly 150.

The transmitter assembly 150 may include a plurality of coils arranged orthogonally to one another to produce a magnetic field in and/or around the anatomical region of interest. It should be noted that, although the transmitter assembly 150 is shown under the body of the patient 112 and under the table 134 in FIG. 1, the transmitter assembly 150 may be placed in another location, such as, attached to the radiation emitter 130, from which the magnetic field generators can project a magnetic field in the anatomical region of interest. In accordance with certain embodiments the transmitter assembly 150 is within the field of view 136. The ECU 126 may control the generation of magnetic fields by transmitter assembly 150.

The position sensors 152 are configured to generate an output dependent on the relative position of the position sensors 152 within the field generated by the transmitter assembly 150. In FIG. 1, the position sensors 152 and the medical tool 116 are shown disposed proximate to the heart 114, for example, the medical tool 116 may be inserted into the coronary sinus, a tributary from the coronary sinus, or the like. The navigation system 120 determines the location of the position sensors 152 within the generated field, and thus the position of the medical tool 116 as well. The navigation system 120 may further determine navigation coordinates, such as a Cartesian coordinate (e.g., (X, Y, Z)), of a navigation coordinate system.

The ECU 126 of the navigation system 120 may include or represent hardware circuits or circuitry that include and/or are connected with one or more logic based devices, such as processors, microprocessors, controllers, microcontrollers, or other logic based devices (and/or associated hardware, circuitry, and/or software stored on a tangible and non-transitory computer readable medium, data storage, or memory). The ECU 126 may receive a plurality of input signals including signals generated by the medical tool 116, the position sensors 152, an operator system interface 154 (e.g., keyboard, touchscreen, graphical user interface, computer mouse, touchpad, or the like), and one or more patient reference sensors (not shown) and generate a plurality of output signals including those used to control the medical tool 116 and/or the display 158. The ECU 126 may also receive an input signal from one or more external physiologic sensors (not shown) configured to measure a physiologic feature of interest from the patient 112, such as cardiac motion, respiratory motion, involuntary motion by the patient 112, or the like. The ECU 126 may adjust the position and/or orientation of the medical tool 116 determined by the navigation system 120 based on the cardiac motion, respiratory motion, involuntary motion of the patient 112 measured by the one or more external physiologic sensors. In at least one embodiment, the ECU 126 may be configured to measure and sort or segregate images from, for example, a medical imaging system 118 based on a timing signal of the external physiologic sensors. For example, ECU 126 may sort images based on the phase of the patient's cardiac cycle at which each image was collected, as more fully described in U.S. Pat. No. 7,697,973, which is hereby incorporated by reference in its entirety.

Optionally, the CNS 110 may include the medical imaging system 118. The CNS 110 may further include a registration system for registering a group of images of the anatomical region of the patient 112 in a navigation coordinate system of the navigation system 120 as generally described and shown in U.S. Patent Publication 2013/0272592 and International Pub. No. WO2012/090148, the entire disclosure of which is expressly incorporated herein by reference.

The medical imaging system 118 may be provided to acquire images of a region and/or organ (e.g., the heart 114) of the patient 112 or another anatomical region of interest. The imaging system 110 may, for example, comprise of a fluoroscopic imaging system. Additionally or alternatively, rather than a fluoroscopic imaging system, computed tomography (CT) imaging systems, a three-dimensional radio angiography (3DRA) system, SPECT, PET, X-ray, MR, ultrasound and the like may be used. Although the medical imaging system 118 is described herein for an exemplary embodiment of the invention, the medical imaging system 118 is not required for the inventive subject matter described within this application.

The medical imaging system 118 may include a C-arm support structure 128, a radiation emitter 130, and a radiation detector 132. The emitter 130 and detector 132 are disposed on opposite ends of the support structure 128 and disposed on opposite sides of the patient 112 as the patient 112 lays on an operation table 134. The emitter 130 and detector 132 define a field of view 136 and are positioned such that the field of view 136 includes the anatomical region of interest as the patient 112 lays on the operation table 134. The medical imaging system 118 is configured to capture images of anatomical features and other objects within the field of view 136. The support structure 128 may have freedom to rotate about the patient 112 as shown by lines 138 and 140. The support structure 128 may also have freedom to slide along lines 142 and 144 (e.g., along the cranio-caudal axis of the patient 112) and/or along lines 146 and 148 (e.g., perpendicular to the cranio-caudal axis of the patient 112). Rotational and translational movement of the support structure 128 yields corresponding rotational and translational movement of the field of view 136. Additionally or alternatively, the navigation system 120 may adjust the navigation coordinates of the position of the medical tool 116 to compensate for changes in the C-arm support structure 128 and respiratory movements of the patient as disclosed in the U.S. Provisional Application No. 61/906,300, entitled, “METHOD TO MEASURE CARDIAC MOTION USING A CARDIOVASCULAR NAVIGATION SYSTEM,” which is expressly incorporated herein by reference in its entirety.

The medical imaging system 118 may acquire a group of images of an anatomical region of the patient 112 by first shifting along lines 142, 144, 146, and/or 148 to place the anatomical region of interest within the field of view 136. Second, the support structure 128 may rotate the radiation emitter 130 and the radiation detector 132 about the patient 112, keeping the anatomical region within the field of view 136. The medical imaging system 118 may capture images of the anatomical region as the support structure 128 rotates, providing a group of two-dimensional images of the anatomical region from a variety of angles. For example, the medical imaging system 118 may acquire a first group of images at an anterio-posterior (AP) angle and a second group of images at a left anterior oblique (LAO) angle of the patient 112. The group of images may be communicated to the ECU 126 for image processing and shown on a display or display 158. Optionally, the display 158 may show multiple angles and/or groups of angles simultaneously. The group of images may comprise a sequence of images taken over a predetermined time period, generating a looped medical image. For example, the medical imaging system 118 may acquire a set of loops each at different angles acquired over a three second time period. It should be notes that the predetermined time period may be greater than or lesser than three seconds. Optionally, the predetermined time period may be based on one or more physiologic measurements (e.g., cardiac motion, respiratory motion) from the physiologic sensors. Additionally or alternatively, the display 158 may continually display one or more loops acquired by the medical imaging system 118.

Additionally, one or more patient reference sensors (not shown) may be on the body of the patient 112, for example, on the chest. The patient reference sensors measure a displacement and orientation of the patient reference sensors relative to a predetermined reference point, such as, the position sensors 152 or the transmitter assembly 150.

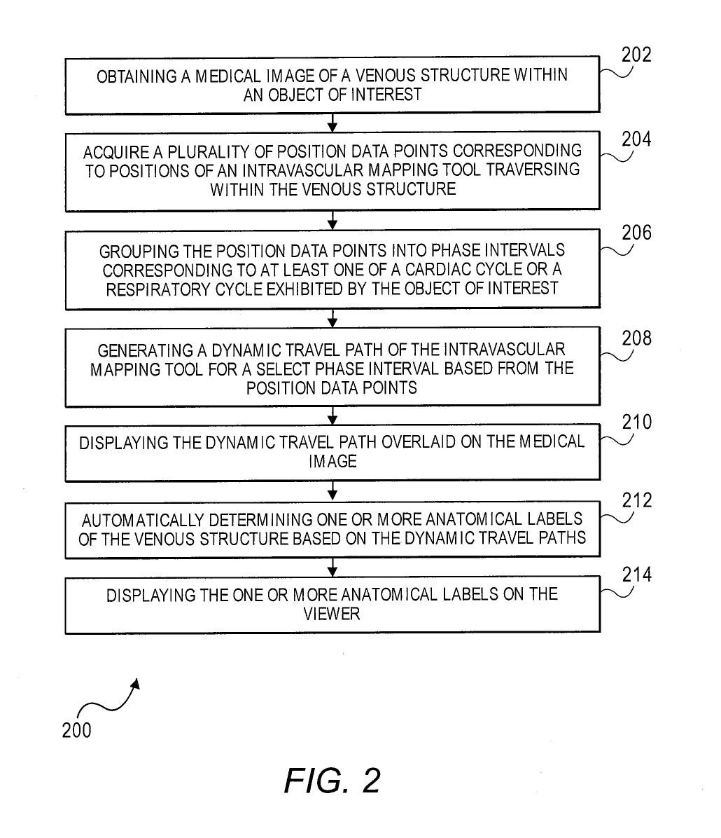

FIG. 2 illustrates a flowchart of a method 200 for generating a virtual venous image. The method 200 may be implemented as a software algorithm, package, or system that directs one or more hardware circuits or circuitry to perform the actions described herein. Optionally, the operations of the method 200 may represent actions to be performed by one or more circuits that include or are connected with processors, microprocessors, controllers, microcontrollers, Application Specific Integrated Circuits (ASICs), Field-Programmable Gate Arrays (FPGAs), or other logic-based devices that operate using instructions stored on a tangible and non-transitory computer readable medium (e.g., a computer hard drive, ROM, RAM, EEPROM, flash drive, or the like), such as software, and/or that operate based on instructions that are hardwired into the logic of the. For example, the operations of the method 200 may represent actions of or performed by a processor when executing programmed instructions stored on a tangible and non-transitory computer readable medium.

At least one technical effect of at least one portion of the methods described herein includes establishing a communication link by i) obtain a medical image of a venous structure within an object of interest, ii) acquiring a plurality of position data corresponding to positions of an intravascular mapping tool traversing within the venous structure, iii) grouping the position data points into phase intervals corresponding to at least one of a cardiac cycle or a respiratory cycle exhibited by the object of interest, iv) generating a dynamic travel path of the intravascular mapping tool for a select phase interval based from the position data points, and v) displaying the dynamic travel path overlaid on the medical image.

Beginning at 202, a medical image (e.g., two dimensional flourograms 600, 601, 900, 901) of a venous structure 300 within an object of interest (e.g., the patient 112) is obtained.

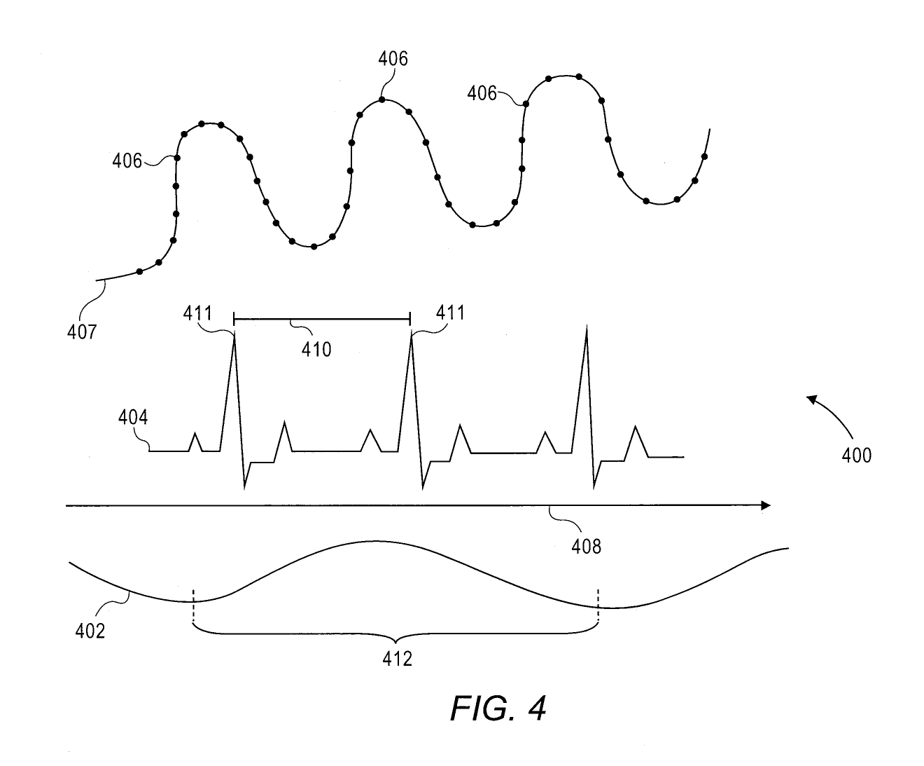

At 204, a plurality of position data points 406 (FIG. 4) are acquired corresponding to a position of an intravascular mapping tool 304 within the venous structure 300.

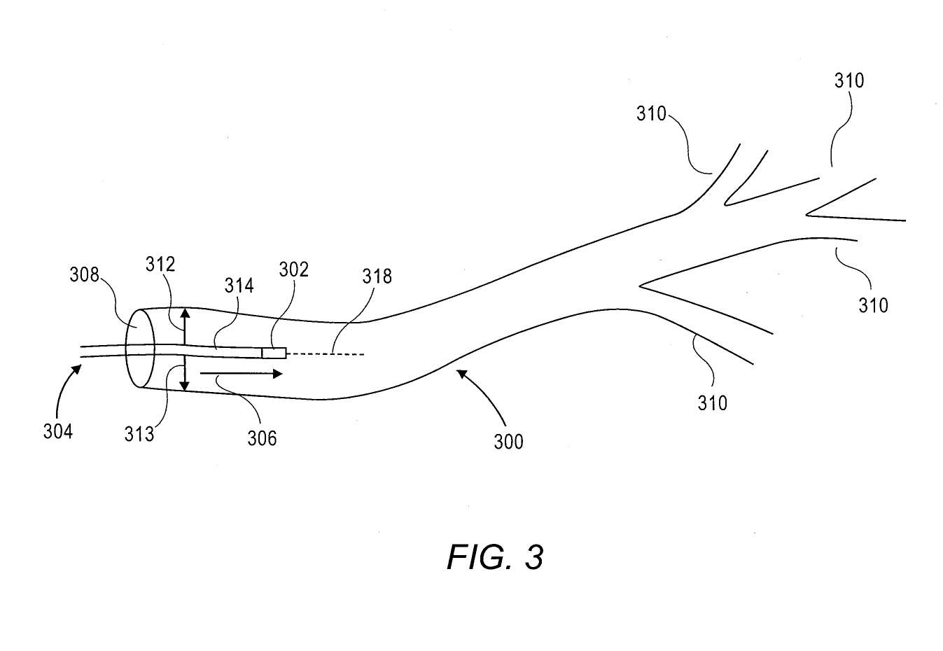

FIG. 3 illustrates the intravascular mapping tool 304 traversing within the venous structure 300. The intravascular mapping tool 304 (e.g., the medical tool 116) may be inserted into the venous structure 300. For example, a cannulation or coronary catheterization may be performed using a catheter (e.g., an outer catheter, diagnostic catheter) allowing access to the venous structure 300, such as the coronary sinus, of an object of interest (e.g., the patient 112). The intravascular mapping tool 304 may be utilized and is configured to be inserted into the venous structure 300 of the object of interest. The intravascular mapping tool 300 may be inserted into the venous structure 300 at an entry point 308 formed during the cannulation. The intravascular mapping tool 304 may traverse or move within the venous structure 300 away from the entry point 308 in the direction of the arrow 306 along a trajectory 318. Additionally or alternatively, the intravascular mapping tool 304 may traverse within one or more venous substructures 310 (e.g., tributaries) connected to the venous structure 300. It should be noted, a diameter of the intravascular mapping tool 304 is less than a diameter of the venous structure 300 and/or one or more venous substructures 310 allowing the intravascular mapping tool 304 to move perpendicular to the arrow 306, such as in the direction of the arrows 312 and 313. Movement of the intravascular mapping tool 304 in the direction of the arrow 312 and 313 further allows the intravascular mapping tool 304 to ricochet, bounce, and/or interact with the inner surface area of the venous structure 300. In connection with FIG. 4, as the intravascular mapping tool 304 traverses within the venous structure 300, a plurality of position data points 406 may be acquired by the navigation system 120 (FIG. 1) for the intravascular mapping tool 304. The plurality of position data points 406 may correspond to a position of the intravascular mapping tool 304 within the venous structure 300.

FIG. 4 illustrates a timing diagram 400 showing cardiac physiologic measurements 404, respiratory physiologic measurements 402, and position data points 406 along an axis of motion (e.g., X axis of the coordinate system, Y axis of the coordinate system, Z axis of the coordinate system) over time (e.g., the horizontal axis 408) acquired by the intravascular mapping tool 304 and one or more physiologic sensors. As the intravascular mapping tool 304 traverses within the venous structure 300, position data may be acquired by the navigation system 120 based on measurements acquired from a position sensor 302. The position data points 406 may be stored on data storage or memory (e.g., a computer hard drive, ROM, RAM, EEPROM, flash drive, or the like). The data storage may be configured to store the position data points 406 collected by the intravascular mapping tool 304 when inserted into the venous structure 300. The position data points 406 form a trajectory 407 of the intravascular mapping tool 304. The position data points 406 may include timing information corresponding to when the position data point 406 was acquired by the intravascular mapping tool 304.

A number of position data points 406 over time may be based on a sampling and/or acquisition frequency of the intravascular mapping tool 304 and/or navigation system 120. For example, the navigation system 120 may have a sampling frequency of thirty hertz thereby thirty position data points 406 may be acquired by the intravascular mapping tool 304 every second.

It should be noted that although the position sensor 302 is positioned on a distal end of the intravascular mapping tool 304, the position sensor 302 may be positioned at other locations on the body 314 of the intravascular mapping tool 304. For example, one or more position sensors 302 may be positioned along the exterior surface of the body 314. The respiratory and/or cardiac physiologic measurements 402, 404 may have been acquired by one or more external physiologic sensors configured to measure a physiologic feature of interest from the patient 112 as described above. Optionally, the cardiac physiologic measurements 404 may have been acquired by additional sensors on the intravascular mapping tool 304, such as an electrophysiological sensor, electrical sensor, or the like.

At 206, the position data points 406 may be grouped into phase intervals corresponding to at least one of a cardiac cycle or a respiratory cycle exhibited by the object of interest. The cardiac cycle corresponds to activity of the heart during systole and diastole activity states. The respiratory cycle or breathing cycle corresponds to the inhalation, inspiratory pause, and exhalation of air entering and exiting lungs of the object of interest.

The cardiac cycles and respiratory cycles are cyclic in nature. The navigation system 120 may identify and/or determine a length of the cardiac cycles and/or the respiratory cycles from the physiologic measurements (e.g., the respiratory physiologic measurements 402, the cardiac physiologic measurements 406) acquired by the one or more physiologic sensors. For example, the navigation system 120 may determine the cardiac cycle based on an R-R interval 410 or heart rate of the patient measured from the R peaks 411 of the cardiac physiologic measurements 406. The respiratory cycle may be measured by movements of the reference sensor corresponding to movements of the chest forming a breathing cycle 412.

The cardiac cycle and the respiratory cycle may be segmented into phase intervals. The phase intervals may correspond to particular activity states of the heart and/or lungs during the cardiac cycle and/or respiratory cycle, respectively. For example, the phase intervals for the cardiac cycle may be segmented into systole, isovolumic contraction, ventricular ejection, isovolumic relaxation, diastole, diastasis, atriol systole, ventricular filling, and/or the like. In another example, the phase intervals for the respiratory cycle may be segmented into inspiration, expiration, pause, and/or the like.

Optionally, the phase intervals may correspond to a morphology (e.g., peak, slope) of the respiratory and/or cardiac physiologic measurements 402, 404. For example, the phase intervals may be based on peaks of the cardiac physiologic measurements 402 corresponding to an atrial depolarization, R-peak 411, repolarization of the ventricles and/or the like. In another example, the phase intervals may be based on peaks of the respiratory physiologic measurements 404 corresponding to inspiration and expiration.

Optionally, the phase intervals may correspond to a time interval within the respiratory and/or cardiac cycle. For example, the navigation system 120 may subdivide the cardiac cycle (e.g., the R-R interval 530) into four segments. Each segments is a time interval equal in length corresponding to one of the phase intervals.

The position data points 406 may be affected through the repetitive cardiac and/or respirator motion of tissue surrounded the intravascular mapping tool 300 caused during the cardiac cycle and/or respiratory cycle. For example, the venous structure 300 and/or the intravascular mapping tool 304 may be shifted and/or moved with respect to the generated field (e.g., generated from the transmitter assembly 150) during a cardiac cycle caused by movement of the heart. In another example, the venous structure 300 and/or the intravascular mapping tool 304 may be shifted and/or moved with respect to the generated field (e.g., generated from the transmitter assembly 150) during the respiratory cycle caused by movement of the lungs or diaphragm.

The navigation system 120 may group the position data points 406 in real-time (e.g., while additional position data points 406 are being acquired) into one or more phase intervals corresponding to the one or more phases of the cardiac cycle and/or a respiratory cycle exhibited by the object of interest. In at least one embodiment, the navigation system 120 may bin or group each position data points 406 corresponding to the timing information of the position data point 406 relative to the phase intervals of the cardiac and/or respiratory motion.

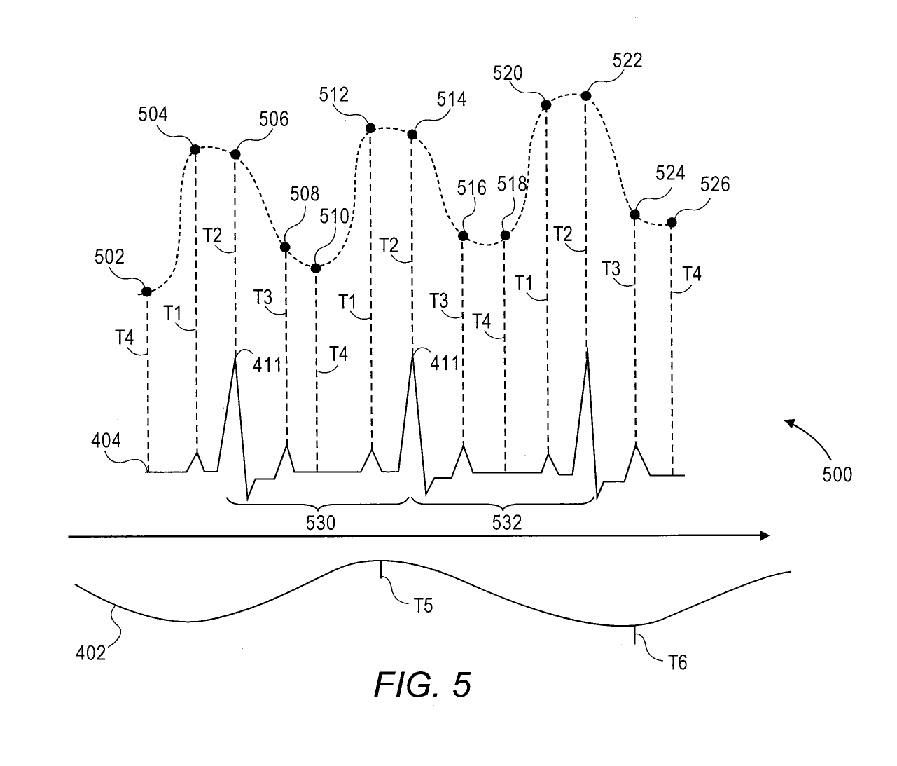

FIG. 5 illustrates a timing diagram 500 showing a subset (e.g., 504-526) of the position data points 406 shown in FIG. 4 grouped into phase intervals, in accordance with an embodiment herein. The cardiac cycle phases correspond to a plurality of phase intervals T1-T4 based on a morphology of the cardiac physiologic measurements 406, such as atrial depolarization (T1), R-peak (T2), repolarization of the ventricles (T3), and a resting state (T4), in each of a plurality of cardiac cycles 530 and 532. Additionally, the respiratory cycle phases corresponds to a plurality of phase intervals T5-T6 based on a morphology of the respiratory physiologic measurements 402, such as inhalation peak (T5) and exhalation peak (T6). The position data points 406 may be matched and are grouped with one of a plurality of the phase intervals T1-T6 according to the timing information of the position data points 406. The grouping indicates that when the positioned sensor 302 of the intravascular mapping tool 304 is at the corresponding position data point 406, the heart and/or lungs of the patient is at the corresponding phase interval T1-T6.

For example, the position data points 504, 512, and 520 shown in FIG. 5 may be grouped into the phase interval T1. The position data points 506, 514, and 522 may be grouped into the phase interval T2. The position data points 508, 516, and 524 may be grouped into the phase interval T3. And the position data points 502, 510, 518, and 526 may be grouped into the phase interval T4.

In at least one embodiment, the timing of the phase intervals of the cardiac and/or respiratory cycles may be offset in relation to the position data points 406. For example, there may be a slight delay or offset between the acquisition of the position data points 406 and the physiologic measurements corresponding to the cardiac cycle and/or respiratory cycle. The navigation system 120 may perform an alignment between the phase intervals to filter and/or remove the offset based on an alignment signal received from the user using the operator system interface 154.

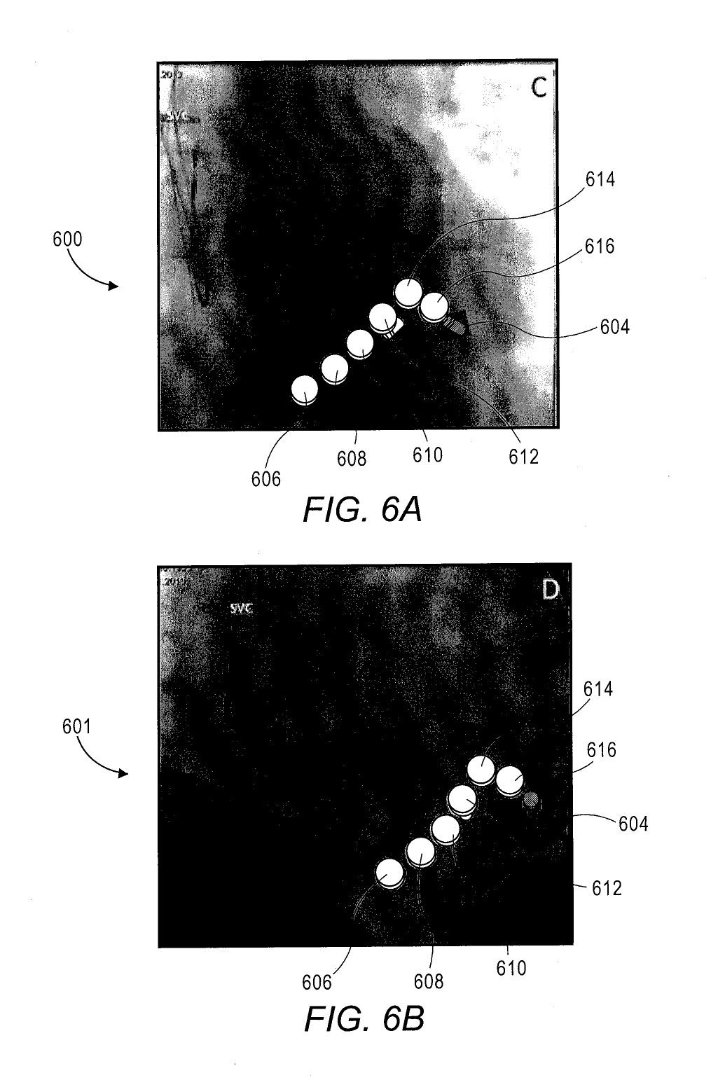

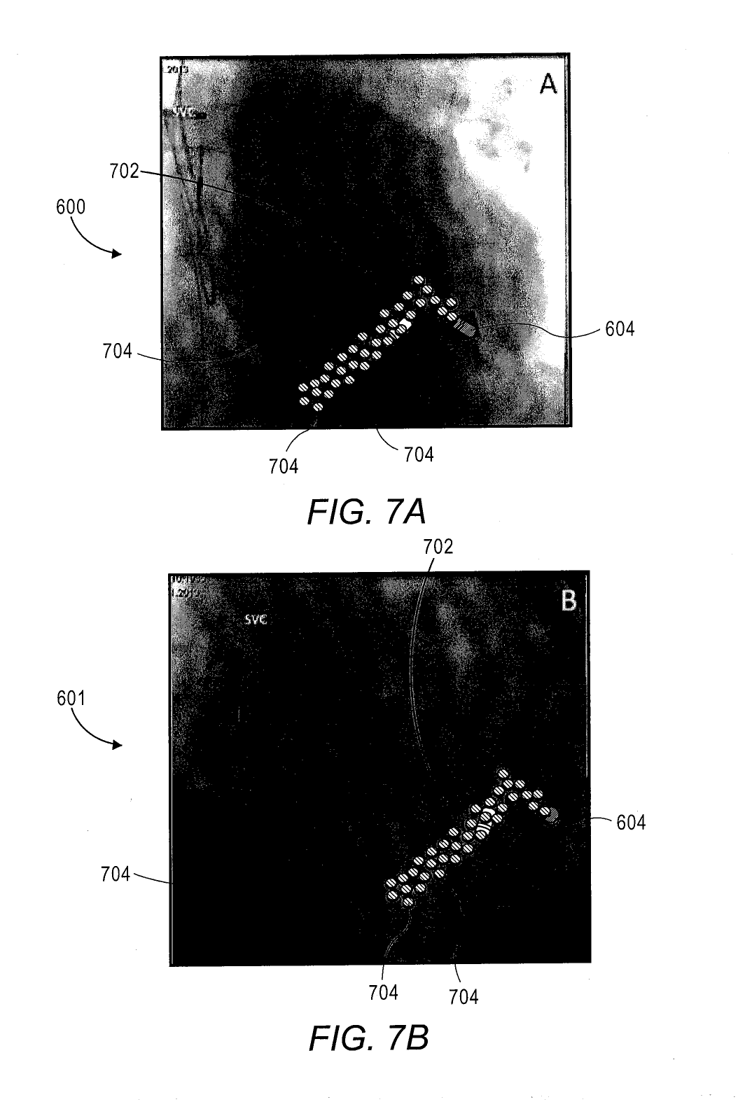

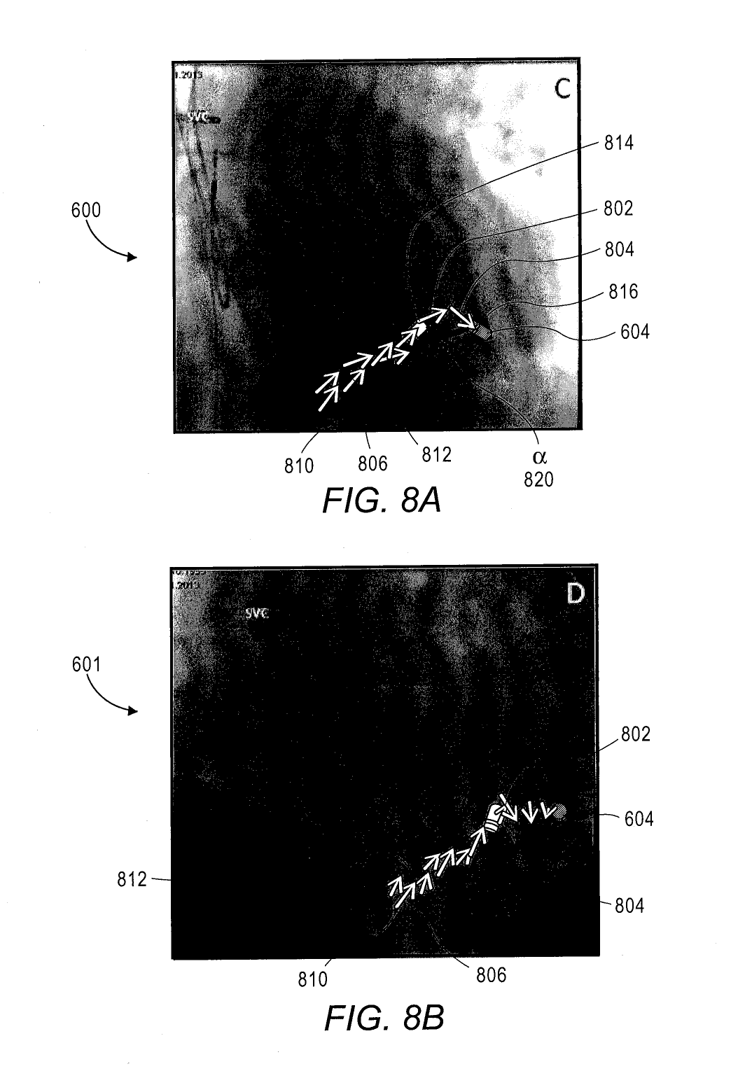

At 208, a dynamic travel path of the intravascular mapping tool 304 for a select phase interval (e.g., selected from one of the phase intervals T1-T7) is generated based from the position data points 406. The dynamic travel path may represent the various positions of the intravascular mapping tool 304 traversing through the venous structure 300 during the select phase interval. For example, the dynamic travel path may be a dynamic point-cloud (e.g., the dynamic point-cloud 702 of FIG. 7) formed by the position data points 406 of the select phase interval. In another example, the dynamic travel path may be a series of landmark icons (e.g., the landmark icons 606-616 of FIG. 6). In another example, the dynamic travel path may be a series of orientation vectors (e.g., the orientation vectors 802-806 of FIG. 8) corresponding to directions and/or orientations of the intravascular mapping tool 304 while traversing within the venous structure 300.

The dynamic travel path is representative of the venous structure 300. For example, the movement of the intravascular mapping tool 304 is bounded and/or limit by the interior of the venous structure 300. Thereby, changes in directions of the intravascular mapping tool 304, ranges (e.g., limits, spatial distances) of the position data points 406, or the like may form a representative volume of the venous structure 300 and/or path of the venous structure 300 within the object of interest.

At 210, the dynamic travel path is displayed being overlaid on the medical image. For example, the dynamic travel path may correspond to a series of landmark icons 606-616.

FIGS. 6A-B illustrate a dynamic travel path visualized as the landmark icons 606-616, as displayed on the display 158, overlaid on two dimensional flourograms 600-601, acquired at different angles. The two dimensional flourogram 600 corresponds to an interior-posterior angle, and the two dimensional flourogram 601 corresponds to a left anterior oblique angle. In at least one embodiment, the navigation system 120 may generate a three dimensional image based on the two dimensional flourograms or looped medical image allowing a user to rotate and/or interact visually with the dynamic travel path within a three dimensional space.

The two dimensional flourograms shown on FIGS. 6A-B may be a subset of a plurality of pre-recorded flourograms over one or more cardiac and/or respiratory cycles, forming a looped medical image. The navigation system 120 may select the landmark icons 606-616 corresponding to the phase interval represented in the two dimensional flourograms 600-601. The navigation system 120 thereby synchronizes positions of the select dynamic travel path with the looped medical image. For example, the navigation system 120 may include a plurality of landmark icons (e.g., in addition to the landmark icons 606-616) for multiple phase intervals that are represented by the looped medical image. The landmark icons 606-616 may correspond to the activity state T4. Additionally, the two dimensional flourograms 600-601 may represent a phase corresponding to the activity state T4. Thus, the navigation system 120 overlays the select dynamic travel path represented by the landmark icons 606-616 when the two dimensional flourograms 600-601 are shown on the display 158, since the landmark icons 606-616 correspond to the phase represented on the looped medical image (e.g., the two dimensional flourograms 600-601) displayed on the display 158. Alternatively, when an alternative phase is represented by the two dimensional flourograms of the looped medical image, the navigation system 120 may overlay landmark icons corresponding in the phase interval corresponding to the alternative phase.

The landmark icons 606-616 delineate a trajectory of the intravascular mapping tool 304 traversing within the venous structure 306. The trajectory enables a virtual depiction of the venous structure 306 on the display. It should be noted that although the landmark icons 606-616 illustrated in FIGS. 6A-B are spherical in shape, in various other embodiments the landmark icons 606-616 may have shapes that are rectangular, triangular, in a cross configuration, and/or the like. Optionally, the display 158 may show a current position icon 604. The current position icon 604 may represent a present and/or current position of the intravascular mapping tool 304 within the venous structure 306.

In various embodiments, the navigation system 120 may select position data points 406 from each phase interval based on a predetermined interval. The selected position data points may form the dynamic travel paths and are displayed as the landmark icons 606-616 on the display 158. For example, each landmark icon 606-616 may correspond to one or more position data points 406 at predetermined intervals previously traveled by the intravascular mapping tool 304. The predetermined interval may be based on a set time period, such as, thirty seconds. For example, the landmark icon 606 may correspond to the position data point 502 representing an entry point (e.g., the entry point 308). The user may designate the predetermined interval via the operator system interface 154 corresponds to ‘n’ seconds. Such that each subsequent landmark icon 608-616 shown on the display 158 may represent a position data point 406 separated and/or acquired ‘n’ seconds after a previous landmark icon 606-616. For example, the landmark icon 608 may correspond to the position data point 526. The position data point 526 is separated from and/or acquired ‘n’ seconds after the position data point 502.

Additionally or alternatively, the predetermined interval may be based on a set spatial distance. For example, the user may designate the predetermined interval via the operator system 154 correspond to ‘n’ millimeters. Such that each subsequent landmark icon 608-616 may represent position data points 406 separated and/or acquired ‘n’ millimeters after a previous landmark icon 606-616.

Additionally or alternatively, the predetermined interval may be based or adjusted based on a velocity or speed of the intravascular mapping tool 304 as the intravascular mapping tool 304 traverses within the venous structure 300. For example, the user may designate a predetermined interval baseline. The predetermined interval baseline may define a corresponding distance and/or time interval for selecting which subsequent landmark icon 608-616 is displayed on the display 158, similarly as described above. The predetermined interval baseline may further include a velocity threshold. The velocity threshold may represent a distance the intravascular mapping tool 304 has traversed within the venous structure 300 or traveled over a unit of time. The velocity threshold may be used by the navigation system 120 to adjust the predetermined interval with respect to the predetermined interval baseline. For example, when a velocity of intravascular mapping tool 304 is above the velocity threshold, the navigation system 120 may automatically decrease the predetermined interval for subsequent landmark icons 606-616. Alternatively, when a velocity of the intravascular mapping tool 304 is below the velocity threshold, the navigation system 120 may automatically increase the predetermined interval for subsequent landmark icons 606-616.

Additionally or alternatively, the predetermined interval may be dynamic corresponding to an input by the user. For example, while operating the operator system interface 154, the user may select and/or activate a capture function. The capture function may instruct the navigation system 120, when selected by the user, to position a landmark icon (e.g., landmark icons 606-616) representing one or more position data points 406 acquire at or about when the capture function was selected and/or activated.

Additionally or alternatively, the dynamic travel paths may correspond to a dynamic point-cloud 702 as shown in FIGS. 7A-B. FIGS. 7A-B illustrate the two dimensional flourograms 600-601 automatically overlaid with a plurality of cloud points 704 forming a dynamic point-cloud 704 displayed on the display 158. The plurality of cloud points 704 shown in the two dimensional flourograms 600-601 may correspond to a subset of cloud points which are displayed at other phases represented by the looped medical image. The cloud points may correspond to the position data points 406 acquired while the intravascular mapping tool 304 traverses within the venous structure 300. For example, each of the cloud points may correspond to position data points 406 which are acquired at predetermined intervals equal to and/or approximate to the sampling frequency (e.g., thirty hertz).

Additionally or alternatively, the navigation system 120 may segment one or more select position data points 406 from the phase intervals based on a predetermined distance of the one or more select position data points with respect to a proceeding position data point. The remaining data points of the phase intervals may be selected to form the dynamic travel paths, for example, the dynamic point-cloud 702. During insertion of the intravascular mapping tool 304 and/or select times while the intravascular mapping tool 304 is traversing within the venous structure 300, one or more positon data points 406 acquired by the intravascular mapping tool 304 may be approximately the same and/or unchanged. The navigation system 120 may select a subset of the position data points 406 for the cloud points 704 forming the dynamic point-cloud. For example, the intravascular mapping tool 304 may be held and/or stopped by the user for three seconds within the venous structure 304 before the user traverses the intravascular mapping tool 304 into one or more of the venous substructures 310. While the intravascular mapping tool 304 is held and/or stopped, position data points 406 are continually acquired at the sampling rate of thirty seconds. Thereby, at the position the intravascular mapping tool 304 was held and/or stopped, the navigation system 120 may have acquired ninety position data points 406, which may be later grouped by the navigation system 120. The navigation system 120 may apply a distance threshold corresponding to a predetermine distance when selecting the position data points 406 that correspond to the cloud points 704. For example, the cloud points 704 may correspond to selected position data points 406 that are positioned at a distance that is equal to and/or greater than the distance threshold relative to a previously acquired position data point 406.

In various embodiments, the navigation system 120 may determine orientation data of the venous structure based on the position data points 406. For example, the navigation system 120 may determine orientation vectors 802-806 that correspond to two or more position data points 406. FIGS. 8A-B illustrate the two dimensional flourograms 600-601 automatically overlaid with a plurality of orientation vectors 802-806. Each of the orientation vectors 802-806 may correspond to two or more position data points 406 acquired while the intravascular mapping tool 304 traverses within the venous structure 300. In various embodiments, the dynamic travels path is formed from the orientation vectors 802-806. A size and overall direction of the orientation vectors 802-806 may correspond to a direction of the intravascular mapping tool 304 while traversing within the venous structure 300.

For example, the orientation vector 806 includes a base 810 and a distal end 812. The base 810 and the distal end 812 may each, respectively, correspond to one of the position data points 406. Particularly, the base 810 may correspond to a position data point 406 acquired previously in time relative to the distal end 812. The orientation vector 806 corresponds to a positional relationship or orientation of the intravascular mapping tool 304 referenced from the base plate 810, particularly the corresponding position data point 406 of the base plate 810, to the distal end 812 within the venous structure 300 corresponding to.

Optionally, the navigation system 120 may determine a take-off angle 820 based on a first and second orientation vector. The take-off angle 820 may correspond to a turn or change in direction of the intravascular mapping tool 300. For example, the take-off angle 820 may correspond to the intravascular mapping tool 304 entering one or more of the venous substructures 310. The take-off angle 820 may be formed with respect to two subsequent orientation angles. For example, the take-off angle 820 is formed with respect to the orientation angles 802 and 804. Particularly, the take-off angle 820 may be formed from a base 814 of the first orientation vector, the orientation vector 802, and an end 816 of the second orientation vector, the orientation vector 804.

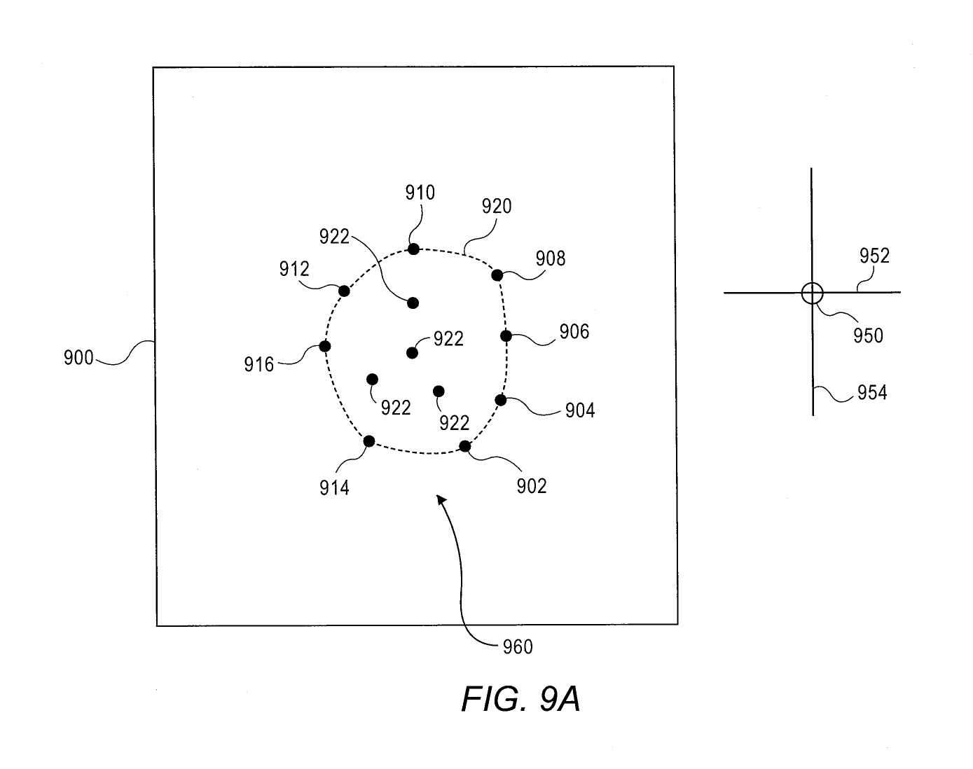

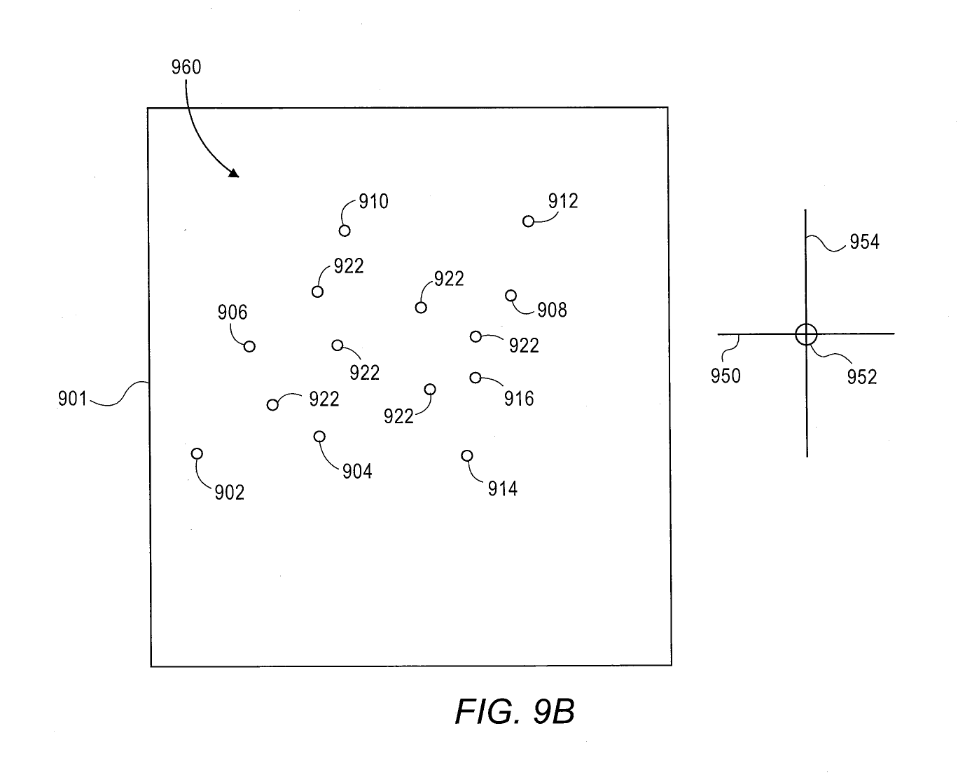

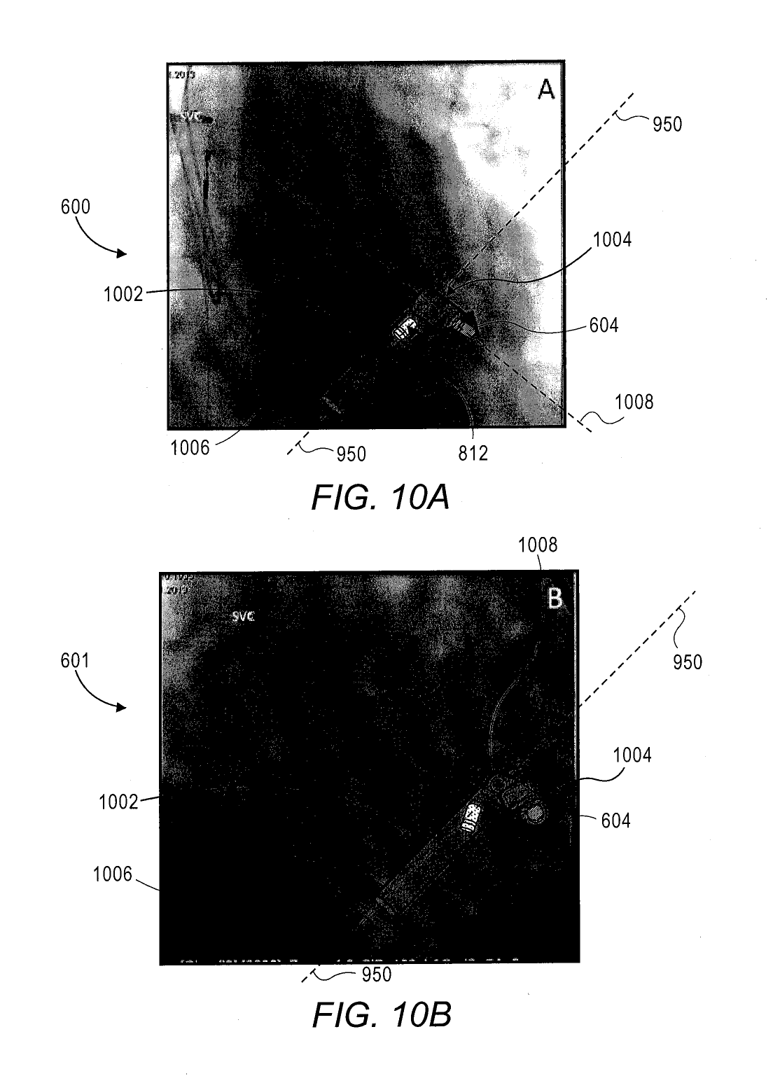

In connection with FIGS. 9A-B and 10A-B, the navigation system 120 may segment interior position data points 922 relative to remaining positon data points 902-916 from the phase intervals, such that the dynamic travel paths form one or more tubular boundaries 1002-1004. Optionally, the navigation system 120 may form the one or more tubular boundaries 1002-1004 in real-time and/or continually updates as the intravascular mapping tool 304 traverse the venous structure 300 such that after enough data acquisitions, a dynamic three dimensional segmentation (e.g., along the axes 950-954) is created that represents the venous structure 300 in a three dimensional space. It should be noted, as shown in FIGS. 10A-B, the dynamic segmentation may be projected or overlaid onto a two dimensional looped medical image, such as the two dimensional flourograms 600-601.

FIG. 9A-B illustrate two dimensional flourograms 900-901 automatically overlaid with a subset 960 of the plurality of cloud points 704. The two dimensional flourograms 900-901 represents alternative angles relative to the two dimensional flourograms 600-601 as shown in FIG. 7. For example, the two dimensional flourogram 900 illustrates a view normal with respect to an axis 950 that is aligned with the trajectory 318 of the intravascular mapping tool 304. Optionally, the axis 950 may be based and/or adjusted based on one or more orientation vectors 802-806. The two dimensional flourogram 900 illustrates a view normal with respect to an axis 952 which is perpendicular to the trajectory 318 of the intravascular mapping tool 304.

As the intravascular mapping tool 304 delineates the boundaries of the venous structure 300 (e.g., along the arrows 312 and 313), the plurality of cloud points 704 may extend along and around the axes 952 and 954. A displacement or radial distance of the cloud points 704, away from the axis 950, is limited by the boundaries of the venous structure 300. The navigation system 120 may determine the boundaries of the venous structure 300 from the cloud points 704 by segmenting the interior position data points (e.g., 922) relative to remaining position data points (e.g., 902-916) from each phase interval.

For example, when the position data points 406 are grouped into phase intervals, the navigation system 120 may determine a displacement or radial distance of the position data points 406 away from a reference axis (e.g., the axis 950) based on the trajectory 318 and/or orientation vectors 802-806 of the intravascular mapping tool 304. The navigation system 120 may compare the radial distances of the position data points 406 within each phase interval that were acquired proximate in time and/or aligned at a slice plane formed by the axis 954, along the reference axis, relative to each other. The navigation system 120 may segment interior position data points 922 from the phase interval that have radial distances less than the remaining position data points 902-916 and/or are enclosed by the remaining position data points 902-916.

FIGS. 10A-B illustrate the two dimensional flourograms 600-601 automatically overlaid with the one or more tubular boundaries 1002-1004 displayed on the display 158. The navigation system 120 may form the dynamic travel path as one or more tubular boundaries 1002-1004 based on the remaining position data points 902-916. For example, the remaining position data points 902-916 may form a surface area 1006 of the tubular boundary 1002. It should be noted that as additional position data points 406 are acquired, the one or more tubular boundaries 1002-1004 may be continually updated in real-time. For example, the surface area 1006 of the tubular boundary 1002 may be adjusted based on one or more newly determined remaining position data points, relative to the previously determined remaining positon data points 902-916.

Additionally or alternatively, the navigation system 120 may determine changes in a direction of the one or more tubular boundaries 1002-1004 based on the take-off angle 812. For example, the navigation system 120 may determine that the tubular boundary 1002 is aligned along the reference axis, such as the axis 950. During acquisition of the position data points 406, the navigation system 120 may determine a trajectory shift of the intravascular mapping tool 304 by the presence of the take-off angle 820, for example corresponding to the intravascular mapping tool 304 entering one or more of the venous substructures 310. The navigation system 120 may adjust the reference axis for segmenting the interior position data points for position data points 406 acquired subsequent to the take-off angle forming the tubular boundary 1004. The reference axis may be adjusted based on the take-off angle 820 such that the reference axis is shifted to the axis 1008.

Returning to FIG. 2, optionally at 212, one or more anatomical labels of the venous structure may be automatically determined based on the dynamic travel path. Additionally or alternatively, the navigation system 120 may receive one or more anatomical labels of the venous structure 300 from a user interface (e.g., the operator system interface 154). The anatomical labels may include the main coronary sinus, a lateral branch, an anterior branch, or the like. The navigation system 120 may compare changes and/or directions of the dynamic travel paths after a location of the cannulation is identified with a pre-determined generic anatomy stored on data storage or memory (e.g., ROM 1104, RAM 1106, hard drive 1108). The pre-determined generic anatomy may include anatomical information for the anatomical labels based on junctures and/or branches leading from a primary anatomical reference, such as the coronary sinus.

For example, the user may identify and/or input, using the operator system interface 154, the cannulation location at the coronary sinus to the navigation system 120. Following the coronary sinus cannulation, the navigation system 120 may determine a first side branch based on a change in orientation corresponding to a take-off angle (e.g., 820). The navigation system 120 may automatically label the first side branch as a “posterolateral branch” or “lateral branch” based on the information of a first side branch of a coronary sinus described by the pre-determined generic anatomy.

In another example, the intravascular mapping tool 304 is returned and/or repositioned within the coronary sinus. The intravascular mapping tool 304 traverses further away from the first side branch and the cannulation point. The navigation system 120 may determine a second change in orientation, such as another take-off angle. The navigation system 120 may automatically label the second side branch as an “anterolateral branch” or “anterior branch” based on the information of a second side branch of a coronary sinus described by the pre-determined generic anatomy.

Optionally at 214, the one or more anatomical labels are displayed on the display (e.g., the display 158). The one or more anatomical labels may be automatically displayed on the looped medical image such as the two dimensional projection screens (e.g., the two dimensional flourograms 600-601) and/or a three dimensional image. Optionally, the user may edit and/or hide the anatomical labels using the operator system interface 154.

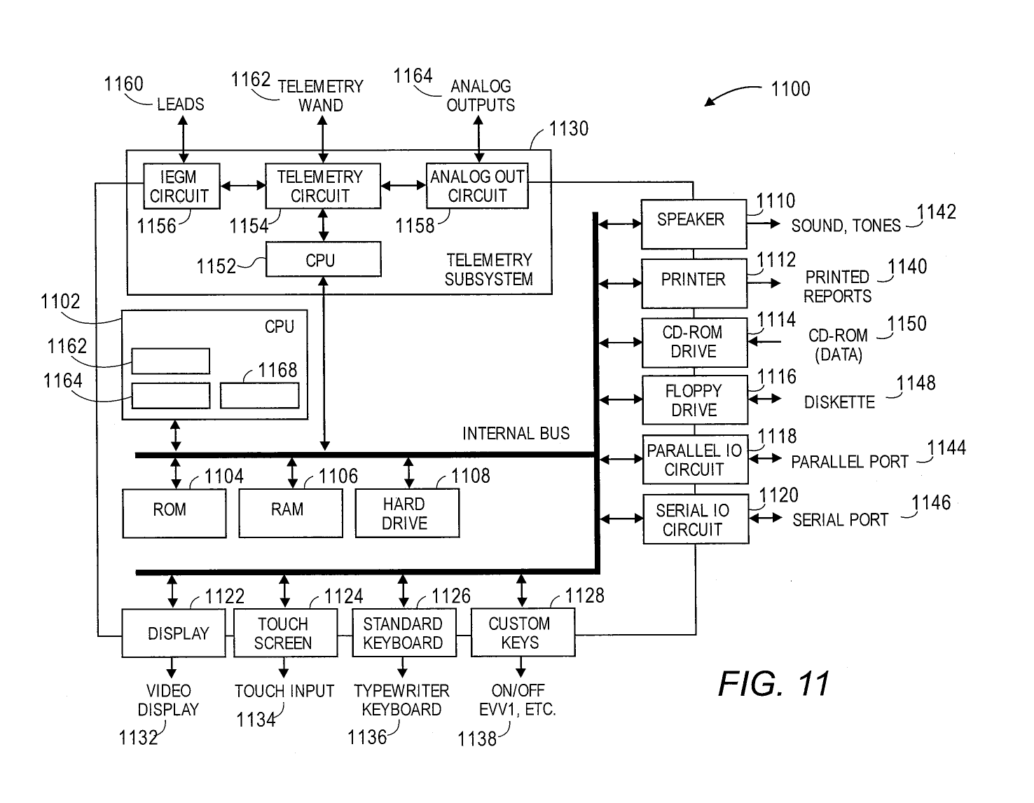

FIG. 11 illustrates a functional block diagram of an embodiment of a navigation system 1100 that is operated in accordance with the processes described herein to generate a virtual venous image, such as a virtual venogram without the use of contrast dye and/or live fluoroscopy. The navigation system 1100 may be a workstation, a portable computer, a PDA, a cell phone and the like. The navigation system 1100 includes an internal bus that connects/interfaces with a Central Processing Unit (CPU) 1402, ROM 1104, RAM 1106, a hard drive 1108, the speaker 1110, a printer 1112, a CD-ROM drive 1114, a floppy drive 1116, a parallel I/O circuit 1118, a serial I/O circuit 1120, the display 1122, a touch screen 1124, a standard keyboard connection 1126, custom keys 1128, and a telemetry subsystem 1130. The internal bus is an address/data bus that transfers information between the various components described herein. The hard drive 1108 may store operational programs as well as data, such as waveform templates and detection thresholds.

The CPU 1102 typically includes a microprocessor, a micro-controller, or equivalent control circuitry, and may interface with the CNS 110. The CPU 1102 may include RAM or ROM memory, logic and timing circuitry, state machine circuitry, and I/O circuitry to interface with the CNS 110. The display 1122 (e.g., may be connected to the video display 1132). The touch screen 1124 may display graphic information relating to the CNS 110. The display 1122 displays various information related to the processes described herein. The touch screen 1124 accepts a user's touch input 1134 when selections are made. The keyboard 1126 (e.g., a typewriter keyboard 1136) allows the user to enter data to the displayed fields, as well as interface with the telemetry subsystem 1130. Furthermore, custom keys 1128 turn on/off 1138 (e.g., EVVI) the navigation system 1100. The printer 1112 prints copies of reports 1140 for a physician to review or to be placed in a patient file, and speaker 1110 provides an audible warning (e.g., sounds and tones 1142) to the user. The parallel I/O circuit 1118 interfaces with a parallel port 1144. The serial I/O circuit 1120 interfaces with a serial port 1146. The floppy drive 1116 accepts diskettes 1148. Optionally, the floppy drive 1116 may include a USB port or other interface capable of communicating with a USB device such as a memory stick. The CD-ROM drive 1114 accepts CD ROMs 1150.

The CPU 1102 is configured to generate a virtual venous image for the CNS 110. The CPU 1102 includes a grouping circuit 1164 that is configured to segmentation analysis circuit module 1164 that is configured to group the position data points into phase intervals corresponding to one or more phases of at least one of a cardiac motion or a respiratory motion of the object of interest. The CPU 1102 also includes a dynamic travel path generation circuit module 1162 that may generate dynamic travel paths of the intravascular mapping tool for each phase interval based on the position data points as described herein. The CPU 1102 also includes an overlay circuit module 1168 that may overlay a select dynamic travel path corresponding to a phase represented on a looped medical image displayed on the display, as explained herein.

The telemetry subsystem 1130 includes a central processing unit (CPU) 1152 in electrical communication with a telemetry circuit 1154, which may communicate with both an IEGM circuit 1156 and an analog out circuit 1158. The circuit 1156 may be connected to leads 1160. The circuit 1156 may also be connected to implantable leads to receive and process IEGM cardiac signals used to determine cardiac motion of the object of interest. Optionally, the IEGM cardiac signals sensed by the leads may be collected by the CNS 110 and then transmitted, to the navigation system 1100, wirelessly to the telemetry subsystem 1130 input.

The telemetry circuit 1154 is connected to a telemetry wand 1162. The analog out circuit 1158 includes communication circuits to communicate with analog outputs 1164. The navigation system 1100 may wirelessly communicate with the CNS 110 and utilize protocols, such as Bluetooth, GSM, infrared wireless LANs, HIPERLAN, 3G, satellite, as well as circuit and packet data protocols, and the like. Alternatively, a hard-wired connection may be used to connect the navigation system 1100 to the CNS 110.

One or more of the operations described above in connection with the methods may be performed using one or more processors. The different devices in the systems described herein may represent one or more processors, and two or more of these devices may include at least one of the same processors. In one embodiment, the operations described herein may represent actions performed when one or more processors (e.g., of the devices described herein) are hardwired to perform the methods or portions of the methods described herein, and/or when the processors (e.g., of the devices described herein) operate according to one or more software programs that are written by one or more persons of ordinary skill in the art to perform the operations described in connection with the methods.

The methods herein may be implemented as a software algorithm, package, or system that directs one or more hardware circuits or circuitry to perform the actions described herein. For example, the operations of the methods herein may represent actions to be performed by one or more circuits that include or are connected with processors, microprocessors, controllers, microcontrollers, Application Specific Integrated Circuits (ASICs), Field-Programmable Gate Arrays (FPGAs), or other logic-based devices that operate using instructions stored on a tangible and non-transitory computer readable medium (e.g., a computer hard drive, ROM, RAM, EEPROM, flash drive, or the like), such as software, and/or that operate based on instructions that are hardwired into the logic of the.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of the inventive subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f), unless and until such claim limitations expressly use the phrase “means for” followed by a statement of function void of further structure.

This written description uses examples to disclose several embodiments of the inventive subject matter and also to enable a person of ordinary skill in the art to practice the embodiments of the inventive subject matter, including making and using any devices or systems and performing any incorporated methods. The patentable scope of the inventive subject matter is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, and the like). Similarly, the programs may be stand-alone programs, may be incorporated as subroutines in an operating system, may be functions in an installed software package, and the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the inventive subject matter are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

What is claimed is:

1. A method for generating a virtual venous image for cardiovascular navigation systems, the method comprising:
obtaining a medical image of a venous structure within an object of interest from a medical imaging system;
acquiring a plurality of position data points by a navigation system for an intravascular mapping tool, wherein the plurality of position data points correspond to a position of the intravascular mapping tool within the venous structure;
grouping the position data points into phase intervals corresponding to at least one of a cardiac cycle or a respiratory cycle exhibited by the object of interest;
generating a dynamic travel path of the intravascular mapping tool for a select phase interval based from the position data points; and
displaying on a display the dynamic travel path overlaid on the medical image.

2. The method of claim 1, wherein the generating operation further comprises segmenting from the position data points of the select phase interval interior position data points, wherein the dynamic travel path forms one or more tubular boundaries.

3. The method of claim 1, wherein the generating operation further comprises selecting a portion of the position data points from the select phase interval based on a predetermined interval, wherein the portion of the position data points form the dynamic travel path and are displayed as landmark icons.

4. The method of claim 3, wherein the predetermined interval corresponds to at least one of a time period, a distance, a speed of the intravascular mapping tool, or an input received from a user interface.

5. The method of claim 1, wherein the generating operation further comprises determining orientation vectors that correspond to two or more position data points, wherein the dynamic travel path is formed from the orientation vectors.

6. The method of claim 5, further comprising determining a take-off angle based on a first and second orientation vector.

7. The method of claim 1, wherein the dynamic travel path corresponds to a dynamic point-cloud.

8. The method of claim 7, wherein the generating operation further comprises segmenting one or more select position data points from the position data points of the select phase interval based on a predetermined distance of the one or more select position data points with respect to a preceding position data point.

9. The method of claim 1, further comprising automatically determining one or more anatomical labels of the venous structure based on the dynamic travel paths; and
displaying the one or more anatomical labels.

10. The method of claim 1, further comprising receiving one or more anatomical labels of the venous structure from a user interface; and
displaying the one or more anatomical labels.

11. A system for generating a virtual venous image comprising:
a display;
an intravascular mapping tool;
a data storage configured to store a plurality of position data points collected by the intravascular mapping tool when inserted into a venous structure of an object of interest, wherein the position data points correspond to a position of the intravascular mapping tool; and
a processor when executing programmed instructions perform the following operations:
obtain a medical image of the venous structure;
group the position data points into phase intervals corresponding to at least one of a cardiac cycle or a respiratory cycle of the object of interest;
generate a dynamic travel path of the intravascular mapping tool for a select phase interval based from the position data points; and
display the dynamic travel path overlaid on the medical image on the display.

12. The system of claim 11, wherein the processor is further configured to segment from the position data points of the select phase interval interior position data points, wherein the dynamic travel path forms one or more tubular boundaries.

13. The system of claim 11, wherein the processor when executing the programmed instructions selects a portion of the position data points from the select phase interval based on a predetermined interval, wherein the portion of the position data points form the dynamic travel path and are displayed as landmark icons on the display.

14. The system of claim 13, wherein the predetermined interval corresponds to at least one of a time period, a distance, a speed of the intravascular mapping tool, or an input received from a user interface.

15. The system of claim 11, wherein the processor when executing the programmed instructions determines orientation vectors that correspond to two or more position data points, wherein the dynamic travel path is formed from the orientation vectors.

16. The system of claim 15, wherein the processor when executing the programmed instructions determines a take-off angle based on a first and second orientation vector

17. The system of claim 11, wherein the predetermined interval corresponds to a dynamic point-cloud.

18. The system of claim 17, wherein the processor when executing the programmed instructions segments one or more select position data points from the position data points of the select phase interval based on a predetermined distance of the one or more select position data points with respect to a preceding position data point.

19. The system of claim 11,
wherein the processor when executing the programmed instructions automatically determines one or more anatomical labels of the venous structure based on the travel path; and
display the one or more anatomical labels on the display.

20. The system of claim 11, further comprising a user interface;
the processor is further configured to:
receive one or more anatomical labels of the venous structure from
the user interface; and display the one or more anatomical labels on the display.
